(19) United States
(12) Patent Application Publication
(10) Pub. No.: US 2021/0268732 A1
(43) Pub. Date: Sep. 2, 2021

(54) Methods And Apparatus For Three-Dimensional Printing Utilizing Croconaine Dyes

(71) Applicants: Chang-Uk Lee, Seattle, WA (US); Andrew J. Boydston, Cross Plains, WI (US); Mark A. Ganter, Edmonds, WA (US); Duane W. Storti, Seattle, WA (US)

(72) Inventors: Chang-Uk Lee, Seattle, WA (US); Andrew J. Boydston, Cross Plains, WI (US); Mark A. Ganter, Edmonds, WA (US); Duane W. Storti, Seattle, WA (US)

(73) Assignee: Ricoh Co., Ltd., Tokyo (JP)

(21) Appl. No.: 16/805,521

(22) Filed: Feb. 28, 2020

(51) Int. Cl. B29C 64/194; B29C 64/153

(52) U.S. Cl. CPC B29C 64/194; B33Y 10/00; B29C 64/153

(57) ABSTRACT

The present invention provides methods, processes, and systems for the manufacture of three-dimensional articles made of polymers using 3D printing. A layer of powder is deposited on a build plate to form a powder bed. Then, a sintering agent is printed on the powder bed in a predetermined pattern. The printed sintering agent is exposed to stimulus which results in the selective sintering of the power printed with the sintering agent. Sequential layers are printed to provide the three-dimensional article. The sintering agent may include a croconaine dye. The sintering agent may further include a surfactant. The three-dimensional object can be cured to produce the three-dimensional article composed of the final polymers.

### FIELD OF INVENTION

The present invention relates to methods and apparatus for creating three-dimensional articles by printing.

### BACKGROUND

Three-dimensional (3D) printing refers to processes that create 3D objects based upon digital 3D object models and a materials dispenser. In 3D printing, a dispenser moves in at least 2-dimensions and dispenses material according to a determined print pattern. To build a 3D object, a platform that holds the object being printed is adjusted such that the dispenser is able to apply many layers of material, and printing many layers of material, one layer at a time, may print a 3D object.

A conventionally known 3D printing process is the UV ink-jet process. It is a three-stage process of applying a material, printing a UV-curable liquid, which is hardened using a UV source. These steps are repeated layer-by-layer. In conventional 3D printing, generally an inkjet type print head delivers a liquid or a colloidal binder material to layers of a powdered build material. The printing technique involves applying a layer of a powdered build material to a surface typically using a roller. After the build material is applied to the surface, the print head delivers the liquid binder to predetermined areas of the layer of material. The binder infiltrates the material and reacts with the powder, causing the layer to solidify in the printed areas by, for example, activating an adhesive in the powder. The binder also penetrates into the underlying layers, producing interlayer bonding. After the first cross-sectional portion is formed, the previous steps are repeated, building successive cross-sectional portions until the final object is formed.

The oldest and the best-known laser-based 3D printing process is stereolithography (SLA). In this process, a liquid composition of a radiation-curable polymer is hardened layer-by-layer by using a laser. A similar process is Selective Laser Sintering (SLS) in which a thermoplastic or a sinterable metal is sintered selectively layer-by-layer by a laser to form the 3D object.

A fused deposition modeling (FDM) process for the production of three-dimensional objects using an extrusion-based, digital manufacturing system has also been used. There are also other known processes that are substantially analogous with slight differences, for example fused filament fabrication (FFF), melt extrusion manufacturing (MEM) or selective deposition modeling (SDM).

In the FDM method, two different polymer filaments are melted in a nozzle and are printed selectively. One of the materials involves a support material, which is needed only at locations above which an overhanging part of the 3D object is printed and requires support during the subsequent printing procedure. The support material can be removed subsequently, e.g. via dissolution in acids, bases or water. The other material (the build material) forms the actual 3D object. Here again, the print is generally achieved layer-by-layer.

### SUMMARY

The present invention provides methods, processes, and systems for manufacture of three-dimensional articles composed of polymers using 3D printing.

In one aspect, disclosed are methods for manufacturing a three-dimensional article, the method comprising depositing a powder on a build plate to form a powder bed; printing, at selected locations on the powder bed, a sintering agent; exposing the printed solution to a stimulus to form a polymer layer of the three-dimensional article; repeating the steps to manufacture remainder of the three-dimensional article; and removing any unbound powder. In one aspect, the sintering agent is a croconaine dye. In additional aspects, the croconaine dye is a water soluble croconaine dye. In further aspects the sintering agent contains a surfactant in combination with a crocoaine dye. Examples of such surfactants include, but are not limited to poly(vinyl alcohol), polyoxyethylene nonylphenyl ether, branched, (IGEPAL CO, but not limited to polyoxyethylene (40) nonylphenyl ether, IGEPAL CO-890), pluronic (poly(ethylene glycol)-block-poly(propylene glycol)-block-poly(ethylene glycol)), polyethylene glycol sorbitan monolaurate (Tween, such as, but not limited to Tween 20), sodium dodecylbenzenesulfonate, and all combinations thereof.

In another aspect, provided are three-dimensional articles made by the process of depositing a powder on a build plate to form a powder bed; printing, at selected locations on the powder bed, a sintering agent; exposing the printed solution to a stimulus to form a polymer layer of the three-dimensional article; repeating the steps to manufacture remainder of the three-dimensional article; and removing any unbound powder. In one aspect, the sintering agent is a croconaine dye. In additional aspects, the croconaine dye is a water soluble croconaine dye. In further aspects the sintering agent contains a surfactant in combination with a crocoaine dye. Examples of such surfactants include, but are not limited to poly(vinyl alcohol), IGEPAL CO-890, pluronic, polyethylene glycol sorbitan monolaurate (Tween, such as, but not limited to Tween 20), sodium dodecylbenzenesulfonate, and all combinations thereof.

In another aspect, a system for printing a three-dimensional article is provided. The system comprising a depositing mechanism to depose a powder layer on a build plate; one or more printing mechanisms to print a sintering agent at selected locations; a stimulus mechanism to provide a stimulus to a printed sintering agent; and a printing controller to repeat the printing mechanism to print the first and second binding agents on a powder layer exposed to a stimulus at a predetermined condition,

These and other aspects of the present invention will become evident upon reference to the following detailed description.

### BRIEF DESCRIPTION OF DRAWINGS

This application contains at least one drawing executed in color. Copies of this application publication with color drawing(s) will be provided by the Office upon request and payment of the necessary fee.

FIG. 1 illustrates a method of printing a three-dimensional article layer by layer as disclosed herein.

FIG. 2 provides a chemical representation of several different croconaine dyes as well as generalized reaction leading to their formation.

FIG. 3 provides a chemical representation of a water soluble croconaine dye.

FIG. 4 illustrates various surfactants that may be used in combination with a croconaine dye.

FIG. 5 is representation of the wavelength absorbance of the croconaine dye of FIG. 3 with various concentrations of poly(vinyl alcohol) (PVA).

FIG. 6 summarizes the effects of various surfactants on the near infrared absorbance of the croconaine dye of FIG. 3.

FIG. 7 depicts temperature during polyethylethylketone (PEEK) with the croconaie dye of FIG. 3 curing as measured by an IR thermal camera in the presence of PVA (FIG. 7A) and in the absence of PVA (FIG. 7B).

### DETAILED DESCRIPTION

### I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise.

The term “alkyl” means the monovalent branched or unbranched saturated hydrocarbon radical, consisting of carbon and hydrogen atoms, having from one to twenty carbon atoms inclusive, unless otherwise indicated. Examples of alkyl radicals include, but are not limited to, methyl, ethyl, propyl, isopropyl, butyl, isobutyl, sec-butyl, tert-butyl, pentyl, n-hexyl, octyl, dodecyl, and the like.

The term “alkylene” as used herein means the divalent linear or branched saturated hydrocarbon radical, consisting of carbon and hydrogen atoms, having from one to twenty carbon atoms inclusive, unless otherwise indicated. Examples of alkylene radicals include, but are not limited to, methylene, ethylene, trimethylene, propylene, tetramethylene, pentamethylene, ethylethylene, and the like.

The term “alkenylene” means the divalent linear or branched unsaturated hydrocarbon radical, containing at least one double bond and having from two to twenty carbon atoms inclusive, unless otherwise indicated. The alkenylene radical includes the cis or trans ((E) or (Z)) isomeric groups or mixtures thereof generated by the asymmetric carbons. Examples of alkenylene radicals include, but are not limited to ethenylene, 2-propenylene, 1-propenylene, 2-butenyl, 2-pentenylene, and the like.

The term “aryl” means the monovalent monocyclic aromatic hydrocarbon radical consisting of one or more fused rings in which at least one ring is aromatic in nature, which can optionally be substituted with hydroxy, cyano, lower alkyl, lower alkoxy, thioalkyl, halogen, haloalkyl, hydroxyalkyl, nitro, alkoxycarbonyl, amino, alkylamino, dialkylamino, aminocarbonyl, carbonylamino, aminosulfonyl, sulfonylamino, and/or trifluoromethyl, unless otherwise indicated. Examples of aryl radicals include, but are not limited to, phenyl, naphthyl, biphenyl, indanyl, anthraquinolyl, and the like.

As used herein, a “build plate” refers to a solid surface made from material such as glass, metal, ceramic, plastic, polymer, and the like.

The term “halogen” as used herein refers to fluoro, bromo, chloro, iodo, or combinations thereof.

The term “optional” or “optionally” means that the subsequently described event or circumstance may or may not occur, and that the description includes instances where the event or circumstance occurs and instances where it does not.

All publications, patents and patent applications cited herein, whether supra or infra, are hereby incorporated by reference in their entirety.

### II. Overview

Disclosed are methods for manufacturing articles made of polymers using three-dimensional printing. The disclosed methods have the advantage of creating removable support features during the 3D printing process. Such support features avoid deformation or fracture of permanent portions of the printed article and are removable through the removal of a binding agent in response to a stimulus once the printing process is complete. The disclosed methods also have the advantage of being able to rapidly print three-dimensional articles that have better mechanical properties, better thermal properties, and the like. The disclosed methods are more flexible than other art methods in that they allow the three-dimensional article to be built quickly with minimal energy input.

In one application, a layer of powder is deposited on a build plate as a powder bed, and then a solution of a sintering agent is selectively printed to appropriate regions of the powder bed in accordance with the three-dimensional article being formed. A stimulus may be applied that heats the sintering agent so as to selectively sinter the powder printed with the sintering agent. Subsequent sequential applications of powder, printing of sintering agent, and exposing to a stimulus, complete the formation of the desired 3D article. The three-dimensional article is thus manufactured layer-by-layer. Once a suitable number of layers have been deposited, the article is cured to provide the three-dimensional article made of the final polymer. The curing can be performed on the build plate or by removing the article from the build plate and then curing it. In one aspect, the sintering agent is a croconaine dye. In additional aspects, the croconaine dye is a water soluble croconaine dye. In further aspects the sintering agent contains a surfactant in combination with a crocoaine dye. Examples of such surfactants include, but are not limited to poly(vinyl alcohol), IGEPAL CO-890, pluronic, polyethylene glycol sorbitan monolaurate (Tween, such as, but not limited to Tween 20), sodium dodecylbenzenesulfonate, and all combinations thereof.

### III. Powder

The three-dimensional form can be made from one or more materials. In certain embodiments, the three-dimensional form is created from a powder that is bound with a binder. Any type of powder can be used to form the three-dimensional form, and the powder can be selected such that the three-dimensional form has the desired properties. Examples of such powders are well known in the art and any such power can be used in the methods described herein. In aspects, the powder can be powdered prepolymer, powdered polymer, powdered ceramic, powdered metal, or powdered plastic. In additional aspects, the powder can be a combination of one or more powdered prepolymers, powdered polymers, powdered ceramics, powdered metals, and powdered plastics.

Examples of prepolymers and/or polymers that may be used include, but are not limited to, thermoplastic polymers, nylon, poly(amic) acids, polyimides, polyketones, such as polyetheretherketone (PEEK), polyaryletherketone (PAEK), polyetherketone (PEK), polyetherketoneketone (PEKK) polyetheretheretherketone (PEEEK), polyetheretherketoneketone (PEEKK), polyetherketoneetheretherketone (PEKEKK), or polyetherketoneketoneketone (PEKKK), reduced form of polyketones, polyethersulfones, and the like. Examples of powdered ceramic that may be used include, but are not limited to, alumina, zirconia, zircon (i.e., zirconium silicate), and silicon carbide-based ceramics. Examples of powdered metals that may be used include, but are not limited to, aluminum, titanium, and iron.

### IV. Sintering Agent

The three-dimensional form can be made from one or more materials. In certain embodiments, the three-dimensional form may comprise particles of powder which have been sintered together. In aspects, a sintering agent is selectively printed on the powder and then exposed to a stimulus which heats the sintering agent sufficient to selectively sinter the powder on which sintering agent has been printed.

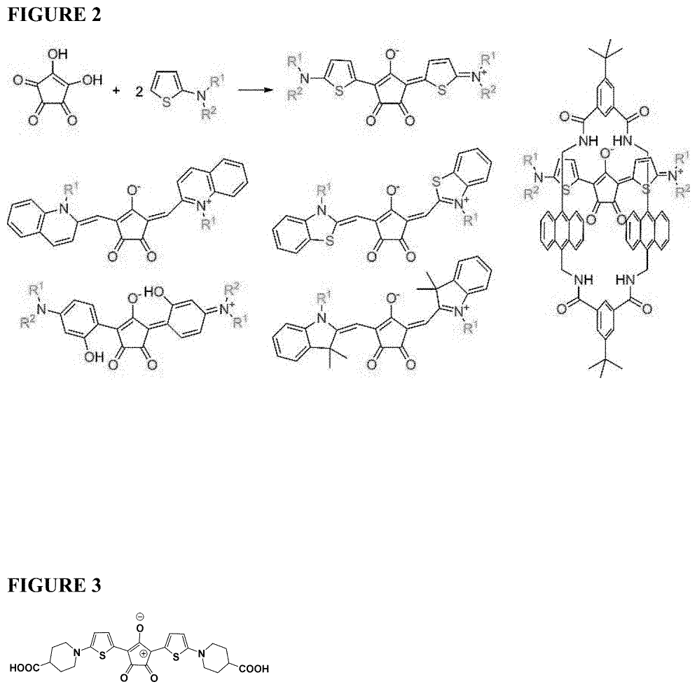

In embodiments, the sintering agent comprises one or more croconaie dyes. Croconaine dyes are photothermal dyes, which convert light to heat. Example of croconaine dyes include, but are not limited to, those croconaine dyes depicted in FIGS. 2 and 3. Croconaine dyes provide various solubility in organic solvents or aqueous media depending on their functional groups. For example, when the functional group is carboxylic acid, a croconaine dye becomes soluble in water (FIG. 3). A water soluble croconaine dye may be prepared by dissolving a croconaine dye in a dilute NaHCO₃ aqueous solution. In particular embodiments, the dilute NaHCO₃ aqueous solution provides a molar ratio of NaHCO₃:croconaine of 1:1, 2:1, 3:1, 4:1, 5:1, 6:1, 7:1, 8:1, 9:1, 10:1, 11:1, 12:1, 13:1, 14:1, 15:1, 16:1, 17:1, 1:18, 19:1, 20:1, or more.

Croconaine dyes tend to be readily aggregated in solution as studied by UV-Vis spectroscopy, showing a broad peak at wavelength (around 710 nm) lower than λₘₐₓ=781 nm. As such, aggregation lowers photothermal efficiency of the croconaine dyes, as less near-IR (NTR) or IR light from, for example but not limited to, a laser, LED, or other source of the proper wavelength of radiation, is absorbed and converted to heat.

In aspects, the photothermal efficiency of croconaine dyes is increased by reducing aggregation of the dye. In one aspect, the aggregation of the croconaine dye can be reduced by including a surfactant in a sintering agent comprising a croconaine dye. Examples of surfactants which may be used include, but are not limited to, poly(vinyl alcohol), IGEPAL CO-890, pluronic, polyethylene glycol sorbitan monolaurate (Tween, such as, but not limited to Tween 20), sodium dodecylbenzenesulfonate, and all combinations thereof.

In embodiments, a sintering agent may comprise a ratio of surfactant:croconaine dye by mass of 1:1, 10:1, 20:1, 30:1, 40:1, 50:1, 60:1, 70:1, 80:1, 90:1, 100:1, 200:1, 300:1, 400:1, 500:1, 600:1, 800:1, 900:1, 1000:1, 1100:1, 1200:1, 1300:1, 1400:1, 1500:1, 1600:1, 1700:1, 1800:1, 1900:1, 2000:1, 3000:1, 4000:1, 5000:1, or more.

In particular embodiments, the surfactant increases the ratio of absorbance intensity at λₘₐₓ (I_{λmax}) to that at shoulder (I_{shoulder}) from 705 nm to 720 nm.

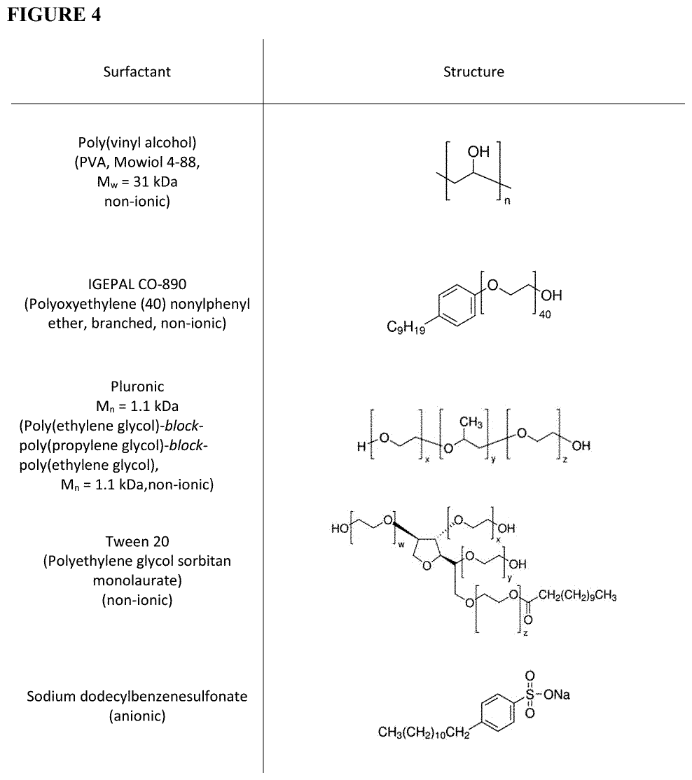

Various surfactants were screened for their ability to reduce aggregations of water-soluble croconaine dye and thus increase absorbance of the proper wavelengths of electromagnetic radiation. FIG. 4 provides non-limiting examples of surfactants with enough solubility in water such that they may be used in conjunction with a water soluble croconaine dye.

An aqueous solution of the croconaine dye was prepared by dissolving in dilute NaHCO₃ aqueous solution (2 equivalents of NaHCO₃ to croc. dye). The surfactants of FIG. 4 were mixed with a croconaine dye aqueous solution in a croconaine dye:surfactant ratio of 1:100, 1:500 or 1:1000 by mass. Effects of surfactants on the aggregations of the croconaine dye was studied by UV-Vis spectroscopy of mixtures of croc. dyes and surfactants.

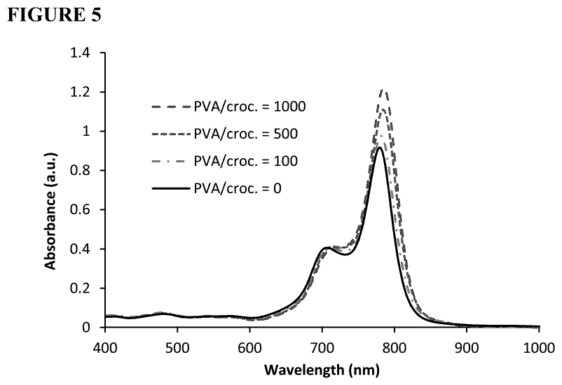

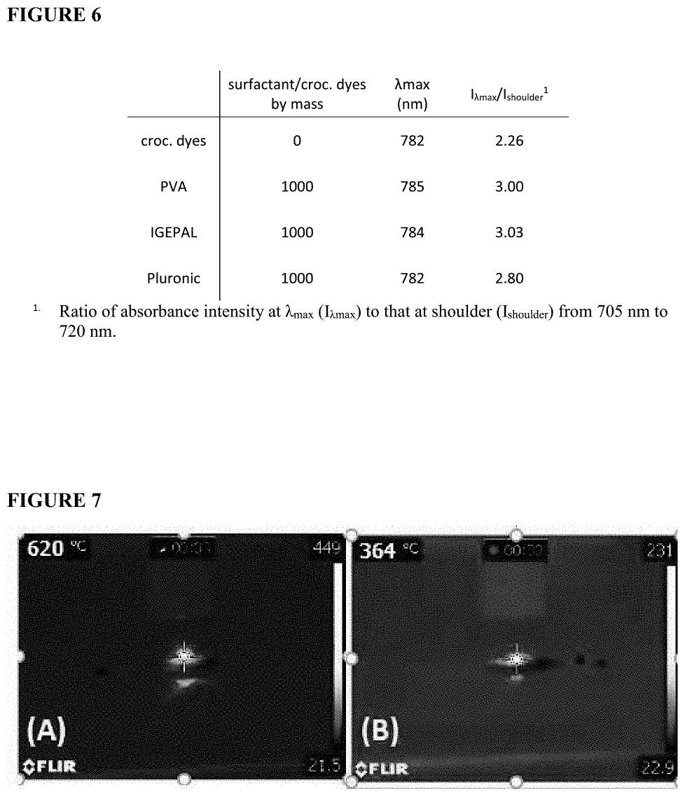

FIG. 5 shows UV-Vis spectroscopy of the water-soluble croconaine dye in water (prepared with 2 equivalents NaHCO₃) with various amounts of one of the surfactants, poly(vinyl alcohol) (PVA). It shows that with increased additions of PVA, the relative intensity of the absorbance peak at 782 nm (to 785 nm) to that of the shoulder peak at 705 nm (to 720 nm) increased. This result demonstrates less aggregations of the dyes in the presence of surfactants compared to the dyes without surfactants. The improved absorbance at 785 nm results in improved photothermal efficiency of croconaine dye when they are imposed to light after jetting on powder bed powder. FIG. 5 summarizes effects of surfactants, PVA, IGEPAL or Pluronic on the NIR absorbance of water-soluble croconaine dye.

The effects of surfactants on photothermal curing of water-soluble croconaine dye were examined. A slurry of PEEK powder and PVA-dye solution (PVA:water-soluble croc. dye=100:1 by weight) was prepared. For a control experiment, a slurry of PEEK powder and croconaine dye was also prepared that lacked a PVA. Light from an 808-nm diode laser was exposed onto the slurry on a glass slide. Temperature during curing was measured by an IR thermal camera. The slurry with PVA and croconaine dye reached 360° C. then 600° C. in 30 seconds (FIG. 7A). Slurry with only croconaine dye reached 360° C. in 60 seconds (FIG. 7B). This result demonstrates that the use of the surfactant PVA improves photothermal efficiency of the water-soluble croconaine dye.

In aspects, the sintering agent is a liquid or may be dissolved in a solvent. The sintering agent, alone, suspended in a carrier, in solution, and/or in the presence or absence of a surfactant, should be of a viscosity which allows deposition by inkjet.

### V. Printing

A powder, a sintering agent as described herein, and a stimulus can be used in a process to create three-dimensional articles using a three-dimensional printing system. A three-dimensional printing system can have a computer, a three-dimensional printer, means for dispensing the powder, and one or more means for dispensing the sintering agent. The three-dimensional printing system can optionally contain a post-printing processing system. The computer can be a personal computer, such as a desktop computer, a portable computer, or a tablet. The computer can be a stand-alone computer or a part of a Local Area Network (LAN) or a Wide Area Network (WAN). Thus, the computer can include a software application, such as a Computer Aided Design (CAD)/Computer Aided Manufacturing (CAM) program or a custom software application. The CAD/CAM program can manipulate the digital representations of three-dimensional articles stored in a data storage area. When a user desires to fabricate a three-dimensional article, the user exports the stored representation to a software program, and then instructs the program to print. The program prints each layer by sending instructions to control electronics in the printer, which operates the three-dimensional printer. Alternatively, the digital representation of the article can be directly read from a computer-readable medium (e.g., magnetic or optical disk) by printer hardware.

Typically, a first layer of the powder can be deposited onto a build plate. The deposited powder is preferably heated to a temperature that is less than about 200° C., and can be in the range of about 30° C. to 170° C., or in the range of about 50° C. to about 150° C. The temperature is selected such that it is below that at which melting, sintering, and/or fusion of the powder occurs. Where the powder is a prepolymer, the temperature may be selected so that it aids in the polymerization of the of the prepolymer when the sintering agent is added. Thus, the deposited powder can be heated to a build temperature of about 40° C., 50° C., 60° C., 70° C., 80° C., 100° C., 110° C., 120° C., 130° C., 140° C., 150° C., 160° C., 170° C., 180° C., 190° C., 200° C., 210° C., 220° C., 230° C., 240° C., 250° C., and the like. The deposited powder can be heated to the desired temperature using any of the known contact or non-contact methods, such as for example, using a heater including, but not limited to, a microwave heater, an infrared heater, an induction heater, a micathermic heater, a solar heater, a heat exchanger, an arc heater, a dielectric heater, a gas heater, a plasma heater, a lamp heater, an infrared heater or any combination thereof, by using a heated plate or a heated roller, or by locally heating the prepolymer solid or powder using a laser or a laser diode, such as, for example, a scanning carbon dioxide laser.

The first layer of the powder can be deposited onto the build plate using any of the known methods, such as, using a roller, using a scraper, using mechanical means, and the like. Thus, for example, a measured quantity of the powder can be distributed over the build plate to a desired thickness using a roller. In another aspect, the layer of the powder can have a thickness of about 0.1 nm to less than 500 nm, of about 5 nm to about 250 nm, of about 0.2 nm to about 100 nm, of about 0.3 nm to about 50 nm, of about 0.3 nm to about 25 nm, of about 0.3 nm to about 20 nm, of about 0.3 nm to about 15 nm, of about 0.3 nm to about 10 nm, of about 0.3 nm to about 5 nm, and the like. In yet another aspect, the layer of the powder can have a thickness of about 10 microns to less than about 500 microns, of about 25 microns to about 250 microns, or of about 50 microns to about 100 microns.

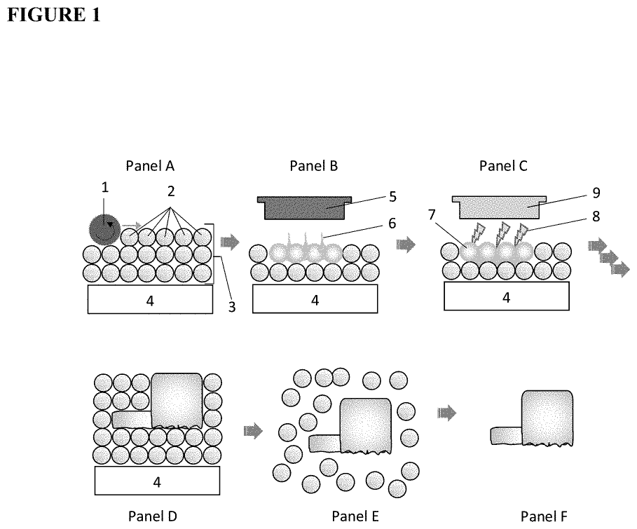

The method of printing a three-dimensional article layer by layer is illustrated in FIG. 1. In Panel A of FIG. 1, the roller 1, deposits powder 2 from one or more powder bed reservoirs to the powder bed 3. The build plate 4 can move in vertical direction as needed. In Panel B of FIG. 1, the head 5 prints a sintering agent 6 on the powder bed 3. The sintering agent can be printed onto the powder bed on the build plate by any printing mechanism. For example, printing may comprise inkjet printing, screen printing, gravure printing, offset printing, flexography (flexographic printing), spray-coating, slit coating, extrusion coating, meniscus coating, microspotting, pen-coating, stenciling, stamping, syringe dispensing and/or pump dispensing the second binding agent in a predefined pattern.

In Panel C of FIG. 1, after the printing of the sintering agent, a permanent structure 7 is formed from the powder and the first binding agent. If required or desired, the sintering agent may be exposed to a stimulus 8 from a stimulus source 9 to bind the powder on which it was deposited. In aspects, the stimulus may be heat, light, enzymes, electromagnetic radiation, oxidation, reduction, acid catalysis, base catalysis, transition metal catalysis, and combinations of any of thereof. In particular aspects the stimulus may be near-infrared or infrared radiation. In aspects the source of the stimulus may be a laser, LEDs, or other source of near-infrared or infrared radiation.

The process of Panels A-C of FIG. 1 may be repeated as desired to build, layer upon layer, a permanent structure and support structures as depicted in Panel D.

After the last layer has been printed, any unbound powder may be removed as is depicted in FIG. 1, Panel E. After removal of any unbound powder, the final product, as depicted in FIG. 1, Panel F is obtained. Thus, a three-dimensional article can be built layer by layer by depositing a series of powder layers on a build plate to form a powder bed, and printing a sintering agent onto the powder bed.

### VIII. Curing

The three-dimensional article obtained using the methods and processes described above can be cured to obtain the final three-dimensional article. The curing of the article can be done while it is attached to the build plate, or the curing of the article can be done by separating it from the build plate first and then curing it. In the curing process, where the powders is polymer or a prepolymer, any unreacted prepolymer is converted to the final polymer. Thus, for example, if the prepolymer is poly(amic acid), the unreacted poly(amic acid) is converted to the polyimide polymer via imidization during the curing process.

In one aspect, during the curing process, poly(amic acid) can be converted to a polyimide polymer by dehydration wherein water is eliminated. Imidization to produce the polyimide, i.e. ring closure in the poly(amic acid), can be effected through thermal treatment, chemical dehydration or both, followed by the elimination of a condensate. The polyimide polymer can be produced by a polymerization/imidization reaction according to a known method such as a thermal imidization by heat treatment accompanied by solvent removal and a chemical imidization, for example, by treatment with acetic anhydride accompanied by solvent removal.

In one aspect, chemical imidization can be used to convert poly(amic acid) to the polyimide. Chemical imidization can be carried out using known agents, such as acetic anhydride; orthoesters, such as, triethyl orthoformate; coupling reagents, such as, carbodiimides, such as dicyclohexylcarbodiimide (DCC) and diisopropylcarbodiimide (DIC), boronic acid, boronic esters, and the like.

In yet another aspect, the curing of compounds such as polyimide and compositions or articles comprising polyimides can be accomplished by curing at elevated temperatures. The curing can be by isothermal heating at a temperature greater than about 190° C., preferably greater than about 250° C., more preferably greater than about 290° C. Thus, the thermal imidization can be carried out at about 280° C., about 290° C., about 300° C., about 310° C., about 320° C., about 350° C., about 375° C., and the like. The curing temperature is selected such that poly(amic acid) is converted to a polyimide and the temperature is below the glass transition temperature or the melting point of the polyimide.

Alternatively, the curing at elevated temperatures can be performed in an isothermal staging process. As an example, such an isothermal staging process can start by heating the material to be cured to 180° C. to 220° C., such as to about 200° C., for some time, typically 1 to 2 hours. However, also less time, such as less than 1 hour, or less than 30 minutes, can be used.

Further, also longer times, such as up to 10 hours may be used. Subsequently, the temperature can be increased in steps. Each step may correspond to an increase of the temperature of 10° C. to 50° C. Further, each step may have duration of 30 minutes to 10 hours, such as 1 to 2 hours. The last step may be curing at a temperature of 250 to 400° C., such as at about 300° C. In an isothermal staging process the duration of each isothermal step may decrease as the temperature increases. A further example of an isothermal staging process, is a process starting at 150° C. in which the temperature is increased by 25° C. every hour until 300° C. is reached.

Curing the final product at elevated temperatures can be performed with continuously increasing temperature. Preferably, the heating rate is slow initially but gradually increased as the temperature increases. Thus, for example, the heating process can start at 150° C. and the temperature is increased continuously until 300° C. or above is reached.

The time of heating for thermal imidization can be about 0.1 h to about 48 h, such as 0.5 h to 15 hours, or 0.5 h to 5 h.

The polyimide polymer thus produced has a tensile strength at break of 150 MPa or higher, more preferably 200 MPa or higher, particularly preferably 250 MPa or higher. The tensile strength can be measured using known methods, such by using the Instron Load Frame instruments.

The polyimide polymer thus produced has a tensile modulus of 1.5 GPa or higher, more preferably 2.0 GPa or higher, particularly preferably 2.5 GPa or higher.

The three-dimensional articles prepared using the methods, processes, and systems of the invention are useful in circuit applications, medical applications, transportation applications, and the like. For example, the three-dimensional articles can be a printed circuit, an insulator, a medical construct such as an orthotic device, a dental implant, prosthetic sockets, and the like, seal rings, washers, and the like.

While the invention has been particularly shown and described with reference to a preferred embodiment and various alternate embodiments, it will be understood by persons skilled in the relevant art that various changes in form and details can be made therein without departing from the spirit and scope of the invention. All printed patents and publications referred to in this application are hereby incorporated herein in their entirety by this reference.

We claim:

1. A method for manufacturing a three-dimensional article, the method comprising:
(a) depositing a powder on a build plate to form a powder bed;
(b) printing, at selected locations on the powder bed, a sintering agent;
(c) exposing the sintering agent to a stimulus so as to selectively sinter the powder printed with the sintering agent; and
repeating steps (a)-(c) to manufacture the remainder of the three-dimensional article wherein the sintering agent comprises a croconaine dye.

2. The method according to claim 1, wherein the croconaine dye is a water soluble croconaine dye.

3. The method according to claim 1, wherein the sintering agent further comprises a surfactant.

4. The method according to claim 3, wherein the surfactant is selected from the group consisting of poly(vinyl alcohol), IGEPAL CO-890, pluronic, polyethylene glycol sorbitan monolaurate, and sodium dodecylbenzenesulfonate.

5. The method according to claim 3, wherein the ratio of surfactant:croconaine dye by mass of is 1:1, 10:1, 20:1, 30:1, 40:1, 50:1, 60:1, 70:1, 80:1, 90:1, 100:1, 200:1, 300:1, 400:1, 500:1, 600:1, 800:1, 900:1, 1000:1, 1100:1, 1200:1, 1300:1, 1400:1, 1500:1, 1600:1, 1700:1, 1800:1, 1900:1, 2000:1, 3000:1, 4000:1, or 5000:1.

6. The method according to claim 1, wherein stimulus comprises near-infrared radiation, infrared radiation, or combination thereof.

7. The method according to claim 1, wherein the powder is selected from the group consisting of prepolymers, polymers, ceramics, metals, and plastics.

8. A system for printing a three-dimensional article, the system comprising:
a depositing mechanism to depose a powder layer on a build plate;
one or more printing mechanisms to a sintering agent at selected locations;
a stimulus mechanism to provide a stimulus to the sintering agent; and
a printing controller to repeat the printing mechanism to print the sintering agent on a powder layer exposed to a stimulus at a predetermined condition;
wherein the sintering agent comprises a croconaine dye.

9. The system of claim 8, wherein the croconaine dye is a water soluble croconaine dye.

10. The system of claim 8, wherein the sintering agent further comprises a surfactant.

11. The system of claim 10, wherein the surfactant is selected from the group consisting of poly(vinyl alcohol), IGEPAL CO-890, pluronic, polyethylene glycol sorbitan monolaurate, and sodium dodecylbenzenesulfonate.

12. The system of claim 10, wherein the ratio of surfactant:croconaine dye by mass of is 1:1, 10:1, 20:1, 30:1, 40:1, 50:1, 60:1, 70:1, 80:1, 90:1, 100:1, 200:1, 300:1, 400:1, 500:1, 600:1, 800:1, 900:1, 1000:1, 1100:1, 1200:1, 1300:1, 1400:1, 1500:1, 1600:1, 1700:1, 1800:1, 1900:1, 2000:1, 3000:1, 4000:1, or 5000:1.

13. The system of claim 8, wherein stimulus mechanism provides a stimulus of near-infrared radiation, infrared radiation, or combination thereof.
